(19) United States
(12) Patent Application Publication
(10) Pub. No.: US 2006/0018973 A1
(43) Pub. Date: Jan. 26, 2006

(54) Composition for stimulating bone-formation and bone consolidation

(76) Inventors: In-San Kim, Taegu (KR); Byung Chae Cho, Taegu (KR)

Correspondence Address: LUCAS & MERCANTI, LLP, 475 PARK AVENUE SOUTH, 15TH FLOOR, NEW YORK, NY, 10016, US

(21) Appl. No.: 10/528,750

(22) PCT Filed: Sep. 30, 2002
(86) PCT No.: PCT/KR02/01837

§ 371 Date: Mar. 22, 2005

(51) Int. Cl. A61K 38/18; A61K 33/42; A61K 31/722

(52) U.S. Cl. 424/603; 514/12; 514/55

(57) ABSTRACT

The present invention relates to a composition for stimulating bone-formation and bone-consolidation, more particularly, to a composition for stimulating bone-formation and bone-consolidation by adding a material for stimulating bone-forming and bone-consolidation to the mixture of tripolyphosphate and water-soluble chitosan. The composition of the present invention can stimulate bone-formation and bone-consolidation in early stages.

### FIELD OF THE INVENTION

The present invention relates to a composition for stimulating bone-formation and bone-consolidation, more particularly; to a composition for stimulating bone-formation and bone-consolidation by adding a material for stimulating bone-forming and bone-consolidation to the mixture of tripolyphosphate and water-soluble chitosan.

### BACKGROUND ART OF THE INVENTION

Bone-loss is often caused by a disease or a car accident recently, so that supplementing bone-loss is importantly required. Bone-transplantation is one way to supplement bone-loss and more preferably bone-filling composition is used. Bone-extension technique is performed today to extend one's height or to correct undersized jaws, for which bone-filling composition is also required a lot.

Bone-extension technique is to stimulate bone-growth, especially growth in height, by stretching based on the theory that “Tension forces stimulate histogenesis”. Bone-extension technique was first devised for the growth of limb bones but has been widely used for jawbone extension. Jawbone extension method is one of techniques performed in the field of cranial jaw facial surgery, which can improve facial ratio not by cutting bone but by moving facial bones gradually by fixing bone-stretching apparatus to retreated parts of jawbone and central facial form.

Bone-extension technique has been successfully used for supplementing the loss of long bone since Ilizarov found out biomechanical elements for bone-extension (Ilizarov G A, J. Dis. Orthop. Inst., 48(1): 1, 1988; Ilizarov G A, Clin. Ortho., 239: 263, 1989; Ilizarov G A, Clin. Ortho., 238: 249, 1989). It is important for performing the successful bone-extension to keep blood circulation in the part of bone-extension well and to fix external fixator stably to both sides of joint part of cortical bone, resulting in the stimulation of bone-consolidation by gradual extension of bone (White S H, J. Bone Join Surgery, 72-B: 350, 1990; White S H, Orthop. Clin. North. Amer., 22: 569, 1991; Fishgrund J., Paley D., Sulter D., Clin. Orthop., 301: 31, 1994).

The period of bone-consolidation depends on extension part of bones such as facial bone or long bone, blood circulation condition, the age of a patient, etc. Bone-consolidation of craniofacial bone takes 3-5 weeks for children and 6-12 weeks for adults after bone-extension, while it takes 3-6 months in long bone regardless of age. Performing bone-extension for craniofacial bone has a couple of problems; one is carrying high possibility of complications and the other is postponing the return to normal life due to the long bone-consolidation time. Precisely, the treatment after bone-extension takes 2-4 months composing of latent phase, bone-extension phase and bon-consolidation phase.

According to Charls and Sailer's report, extending 1 mm a day shows stronger biochemical and physiological characteristics than extending 2-3 mm a day (Carls & Sailer, J. Craniomaxillofac Surg., 94: 152, 1994). Ilizarov has also reported that extending 1 mm a day showed best results while extending 0.5 mm a day caused premature bone-consolidation and extending 2 mm a day caused undesirable changes in extended tissues (Ilizarov, J. Dis. Orthop. Inst., 48 (1): 1, 1988; Ilizarov, Clin. Ortho 239: 263, 1989). In addition, it has also been known that consecutive extending causes the least damage in tissues but the best development of capillary vessels and bone-formation. Therefore, shortening the period of bone-extension and bone-consolidation can contribute to prevent possible complications and to make a patient return to normal life early. In order to shorten the period of bone-extension and bone-consolidation, bone-filling composition is used to stimulate bone-formation and bone-consolidation.

Meanwhile, autobone-graft, treated homograft, heterograft and bone graft substitute have been known to stimulate bone-formation. Autobone-graft is used for the treatment of joint-agglutination or non-agglutinational fracture, or for avoiding damage and void caused by infection, tumor and operation by supplementing bone cavity or bone loss. Transplanted autobone is well adsorbed, resulting in re-circulation of blood. At this time, osteoprogenitor cells are differentiated to bony osteogenesis cells and the activation thereof stimulates bone-regeneration as well as treats bone-loss. However, autobone-graft has problems such as limitation in the amount of extraction and high morbidity caused by the secondary operation for the part of donation. Thus, bone morphogenic protein or other bone-grafting substitute is used to induce bone-regeneration at extended sites. Bone morphogenic protein is regarded as the strongest bone-inducing material but is limited in clinical use because it is very expensive and hard to obtain.

Thus, the present inventors have been tried to find out a composition to stimulate bone-formation and bone-consolidation that is inexpensive and suitable for human. As a result, we, the present inventors, have prepared a composition by adding a material stimulating bone-formation and bone-consolidation to the mixture of tripolyphosphate and water soluble chitosan, and have completed the present invention by confirming that the composition stimulates bone-consolidation in the early phase and shortens the period of bon-generation by cutting bone-consolidation time.

### SUMMARY OF THE INVENTION

The present invention relates to a composition for stimulating bone-formation and bone-consolidation, more particularly, to a composition for stimulating bone-formation and bone-consolidation by adding a material for stimulating bone-formation and bone-consolidation to the mixture of tripolyphosphate and water-soluble chitosan. The composition of the present invention can stimulate bone-formation and bone-consolidation in the early stage.

### BRIEF DESCRIPTION OF THE DRAWINGS

FIG. 1 is a photograph showing the performance of vertical osteotomy at left jaw of a dog for bone-extension with chitosan, β ig-h3 and human bone morphogenic protein (BMP-4),

FIG. 2 is a photograph showing a dual syringe containing water-soluble chitosan, water-soluble chitosan containing β ig-h3, water soluble chitosan containing BMP-4, and 5% tripolyphosphate,
A: 0.5 cc of water soluble chitosan, water soluble chitosan containing β ig-h3, or water soluble chitosan containing BMP-4
B: 0.5 cc of 5% tripolyphosphate

FIG. 3 is a photograph showing a dog in the death-imminent state, seven weeks after bone extension,

FIG. 4A is a photograph showing the extent of bone-consolidation in a control group that was measured with radio-assay five weeks after bone extension, particularly the control group was treated with only tripolyphosphate and was induced to have 2.0 mm extension per day for five days for the group,

FIG. 4B is a photograph showing the extent of bone-consolidation in a control group that was measured with radio-assay seven weeks after bone extension, particularly the control group was treated with only tripolyphosphate and was induced to have 2.0 mm extension per day for five days,

FIG. 5A is a photograph showing the extent of bone-consolidation in a chitosan group that was measured with radio-assay 4 weeks after bone extension, particularly the group was treated with chitosan and tripolyphosphate, and was induced to have 2.0 mm extension per day for 5 days,

FIG. 5B is a photograph showing the extent of bone-consolidation in a chitosan group that was measured with radio-assay 7 weeks after bone extension, particularly the group was treated with chitosan and tripolyphosphate, and was induced to have 2.0 mm extension per day for 5 days,

FIG. 6A is a photograph showing the extent of bone-consolidation in a β ig-h3 group that was measured with radio-assay 4 weeks after bone extension, particularly the group was treated with water-soluble chitosan containing β ig-h3 and tripolyphosphate, and was induced to have 2.0 mm extension per day for 5 days,

FIG. 6B is a photograph showing the extent of bone-consolidation in a β ig-h3 group that was measured with radio-assay 7 weeks after bone extension, particularly the group was treated with water-soluble chitosan containing β ig-h3 and tripolyphosphate, and was induced to have 2.0 mm extension per day for 5 days,

FIG. 7A is a photograph showing the extent of bone-consolidation in a BMP-4 group that was measured with radio-assay 4 weeks after bone extension, particularly the group was treated with water-soluble chitosan containing BMP-4 and tripolyphosphate, and was induced to have 2.0 mm extension per day for 5 days,

FIG. 7B is a photograph showing the extent of bone-consolidation in a BMP-4 group that was measured with radio-assay 7 weeks after bone extension, particularly the group was treated with water-soluble chitosan containing BMP-4 and tripolyphosphate, and was induced to have 2.0 mm extension per day for 5 days,

FIG. 8A is a graph showing the mineral density of bone 4 weeks after bone extension for each group that was injected with chitosan only, water soluble chitosan containing β ig-h3 and water soluble chitosan containing BMP-4 respectively, and was induced to have 2.0 mm extension per day for 5 days,

FIG. 8B is a graph showing the mineral density of bone 7 weeks after bone extension for each group that was injected with chitosan only, water soluble chitosan containing β ig-h3 and water soluble chitosan containing BMP-4, and was induced to have 2.0 mm extension per day for 5 days,

FIG. 9A is a photograph showing the histological section of a control group 4 weeks after bone extension, particularly the group was injected with tripolyphosphate only and was induced to have 2.0 mm extension per day for 5 days,
Arrows: the first cutting parts

FIG. 9B is a photograph showing the histological section of a control group 7 weeks after bone extension, particularly the group was injected with tripolyphosphate only and was induced to have 2.0 mm extension per day for 5 days,
Arrows: the first cutting parts

FIG. 9C is a photograph showing the new bone-formation at the edge of bone extension region in a control group injected tripolyphosphate only and induced to have 2.0 mm extension per day for 5 days, which was confirmed by hematoxylin & eosin staining,
A: fibrous tissue, B: osteoblasts

FIG. 10A is a photograph showing the histological section of a BMP-4 group 4 weeks after bone extension, particularly the group was injected with water soluble chitosan containing BMP-4 and tripolyphosphate, and was induced to have 2.0 mm extension per day for 5 days,
Arrows: the first cutting parts

FIG. 10B is a photograph showing the histological section of a BMP-4 group 7 weeks after bone extension, particularly the group was injected with water soluble chitosan containing BMP-4 and tripolyphosphate, and was induced to have 2.0 mm extension per day for 5 days,
Arrows: the first cutting parts

FIG. 10C is a photograph showing that the center of the extended region was filled with osteoblasts and fibrous tissues, which was confirmed by hematoxylin & eosin staining with the histological section of A,

FIG. 10D is a photograph showing that the center of the extended region was filled with osteoblasts and fibrous tissues, which was confirmed by hematoxylin & eosin staining with the histological section of B,

FIG. 11A is a photograph showing the histological section of a β ig-h3 group 4 weeks after bone extension, particularly the group was injected with water soluble chitosan containing β ig-h3 and tripolyphosphate, and was induced to have 2.0 mm extension per day for 5 days,
Arrows: the first cutting parts

FIG. 11B is a photograph showing the histological section of a β ig-h3 group 7 weeks after bone extension, particularly the group was injected with water soluble chitosan containing β ig-h3 and tripolyphosphate, and was induced to have 2.0 mm extension per day for 5 days,
Arrows: the first cutting parts

FIG. 11C is a photograph showing the new bone-formation over the whole extended region, which was confirmed by hematoxylin & eosin staining with the histological section of B,

FIG. 12A is a photograph showing the histological section of a chitosan group 4 weeks after bone extension, particularly the group was injected with chitosan and tripolyphosphate, and was induced to have 2.0 mm extension per day for 5 days,
Arrows: the first cutting parts

FIG. 12B is a photograph showing the histological section of a chitosan group 7 weeks after bone extension, particularly the group was injected with chitosan and tripolyphosphate, and was induced to have 2.0 mm extension per day for 5 days,
Arrows: the first cutting parts

FIG. 12C is a photograph showing the new bone-formation over the whole extended region, which was confirmed by hematoxylin & eosin staining with the histological section of A,

### DETAILED DESCRIPTION OF THE INVENTION

To achieve the above object, the present invention provides a composition for stimulating bone-formation and bone-consolidation prepared by adding a material stimulating bone-formation and bone-consolidation to the mixture of tripolyphosphate and water-soluble chitosan.

The present invention also provides a use thereof for stimulating bone-formation and bone-consolidation in the early phase.

Further features of the present invention will appear hereinafter.

The present invention provides a composition for stimulating bone-formation and bone-consolidation prepared by adding a material stimulating bone-formation and bone-consolidation to the mixture of tripolyphosphate and water-soluble chitosan.

The composition for stimulating bone-formation and bone-consolidation of the present invention is prepared by adding a material stimulating bone-formation and bone consolidation to the mixture of tripolyphosphate and water-soluble chitosan. β ig-h3, bony morphogenic protein, TGF-β, FGF, IGF-1 and PDGF are the examples for the material to stimulate bone-formation and bone consolidation, but the examples are not always limited thereto. In the preferred embodiments of the present invention, β ig-h3 and BMP-4 were used as materials to stimulate bone-formation and bone-consolidation.

Chitosan is a kind of polysaccharide obtained by deacetylation of chitin, an exoskeleton-structure material of sea Crustacea (Kind, G. M., Bind, S. D., Staren, E. D., Templeton, A. J. and Economou, S. G., Curr. Surg., 47: 37, 1990; Hauschks, P. V., Bone, Vol. 1, 103, 1990, London CRC press; Cunningham, N. S., Paralkar, V. and Reddi, A. H., Proc. Nat. Acad. Sci., 89: 11740, 1982; Malette, W. G., Quigley, H. J. and Adickes, E. D., Nature and Technology, 435: 1986, New York Plenum Press). Muzarelli, etc have disclosed that chitosan introduced to bone-deficient region stimulated normal bone-formation (Muzzarelli, R. A., Mattioli-Belmonte, M., Tiets, C., Biagini, R., Feioli, G., Brunelli, M. A., Fini, M., Giardino, R., Ilari, P. and Biagini, G., Biomaterials, 15: 1075, 1994), and Klokkevold, etc also have reported that chitosan stimulated differentiation of bony osteogenesis cells and induced bone-formation itself (Klokkevold, P. R., Vandemark, L., Kenney, E. B. and Bernard, G. W., J. Periodontol., 67: 1170, 1996).

TGF-β is known to evoke proliferation and differentiation of osteoblasts, especially β ig-h3 is believed to increase the production of various bone intracellular proteins in vitro and decrease the collagen degradation in osteoblasts (Sporn, M. B., Roberts A. B., Springer-Verlag, New York: 3, 1990; Centrella, M., McCarthy, T. L. and Canalis, E., J. Bone Join. Surg., 73(Am): 1418, 1991; Mustoe, T. A., Pierce, G. F., Thomason, A., Gramates, P., Sporn, M. B. and Deuel, T. F., Science, 237: 1333, 1987; Noda, M. and Camilliere, J. J., Endoclinol., 124: 2991, 1989; Joyce, M. E., Jinguski, S., Roberts, A. B., Sporn, M. B. and Bolander, M. E., J. Bone Miner. Res., 4: 225, 1989; Hock, J. M., Canalis, E. and Centrella, M., Endoclinol., 126: 421, 1990; Beck, L. S., Ammann, A. J., Aufdemorte, T. B., DeGuzman, L., Xu, Y., Lee, W. P., McFatridge, L. A. and Chen, T. L., J. Bone Miner. Res., 6: 961, 1991). Among many growth factors, transforming growth factor β (TGF-β) is particularly important regulator for bone-regeneration and development. TGF-β 1 is a strong chemoattractant of osteoblast and leads division of proto-cells of osteoblasts during the endochondrial ossification process. As a cell-attaching protein whose expression is induced by TGF-β, β ig-h3 has functions of attaching and spreading cells by working with integrin (Jung-Eun Kim, Song-Ja Kim, Byung-Heon Lee, Rang-Woon Park, Ki-San Kim and In-San Kim, J. Biol. Chem., 275: 30907-30915, 2000) as well as curing a cut. It is also known to play an important role in the early phase of osteogenesis (Dieudonne, S. C., Kerr, J. M., Xu, T., Sommer B., DeRubeis, A. R., Kuznetsov, S. A., Kim, I-S., Robey, P. G., and Young M. F., J. Cell. Biochem., 76: 231-243, 1999).

Bone morphogenic protein (referred as “BMP” hereinafter) is a bone-forming material and was first found by Urist. BMP has been reported to stimulate pluripotential cells to be differentiated into chondrocytes and osteogenesis cells, and also play an important role in bone-regeneration (Urist, M. R., Science, 150: 893, 1965; Urist, M. R. and Strates, B. S., J. Dent. Res., 50: 1392, 1971; Wozney, J. M., Butterworth Heinermann 1st Ed. London: 397-411, 1994; Wozney, J. M., Mol. Reprod. Dev., 32; 160, 1992; Wozney, J. M., Rosen, V. and Celeste, A. J., Science, 242: 1528, 1988; Ono, I., Tatashita, T., Takita, H. and Kuboki, Y., J. Craniofac. Surg., 7: 418, 1996). 13 human BMPs have been confirmed so far and especially human bone morphogenic protein-4 (BMP-4) has been reported to have excellent effect on bone-regeneration (Boyne, P. J., Bone, 19: 83s, 1996; Zegzula, H. D., Buck, D. C., Brekke, J., Wozney, J. M. and Hollinger, J. O., J. Bone Join. Surg., 79: 1778, 1997; Sporn, M. B., Roberts A B (eds.), Springer-Verlag, New York: 3, 1990).

Tripolyphosphate is immediately hardened when water-soluble chitosan is added thereto. Thus, it is required in this invention to inject chitosan, water soluble chitosan containing β ig-h3, a material to stimulate bone-formation and bone-consolidation, or bone morphogenic protein simultaneously into the same spot using dual syringe shown in FIG. 2 in order to induce immediate solidation at the spot, resulting in preventing injected materials from being transferred to other areas.

For the composition for stimulating bone-formation and bone-consolidation of the present invention prepared by adding β ig-h3 or bone morphogenic protein as a material to stimulate bone-formation and bone-consolidation to the mixture of tripolyphosphate and water soluble chitosan, tripolyphosphate and water soluble chitosan are preferably mixed in the proportion of 20:80˜80:20 weight % and more preferably 50:50 ratio. As a material to stimulate bone-formation and bone-consolidation, β ig-h3 is preferably added to the composition with the amount of 100 μg/ml˜1 μg/ml and more preferably with 300 μg/ml˜600 μg/ml. BMP is also preferably added to the composition with the amount of 50 ng/ml˜500 ng/ml and more preferably with 100 ng/ml˜300 ng/Ml.

The present invention also provides a use of the composition for stimulating bone-formation and bone-consolidation in the early phase.

In order to confirm whether the composition can be used for stimulating bone-formation and bone-consolidation, the present inventors first investigated the effect of the composition prepared by adding water soluble chitosan, water soluble chitosan containing β ig-h3 or water soluble chitosan containing BMP to tripolyphosphate on bone-formation and bone-consolidation as bone-extension operation was performed at the jaw of a dog.

As a result, in the bone sample of control group obtained 4 weeks and 7 weeks after bone extension, extended area was proved to be solid but a little flexible when being bended. As for BMP-4 group prepared by adding water soluble chitosan containing BMP-4 to tripolyphosphate, β ig-h3 group prepared by adding water soluble chitosan containing β ig-h3 to tripolyphosphate and chitosan group prepared by adding just water soluble chitosan to tripolyphosphate, extended bone samples taken 4 weeks after bone extension were proved to be more solid than that of control group taken 7 weeks after bone extension. 7 weeks later, new bones were formed and the extended areas became very solid in those groups.

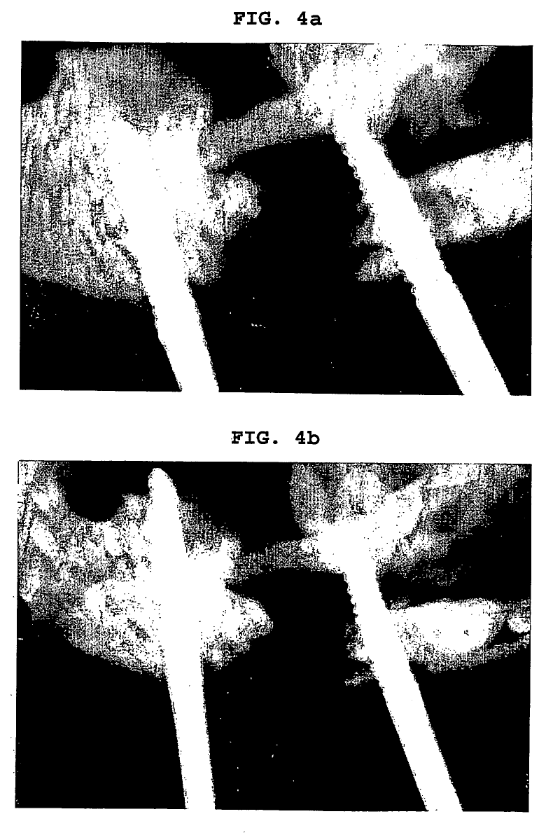

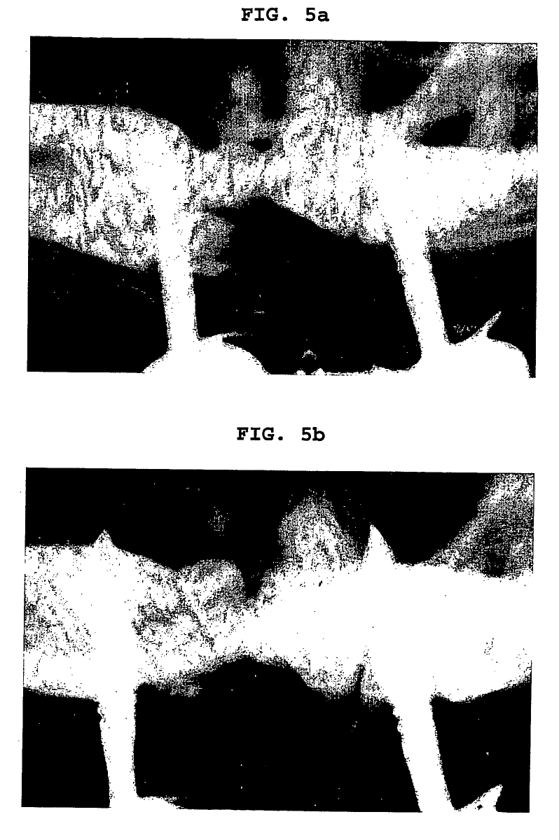

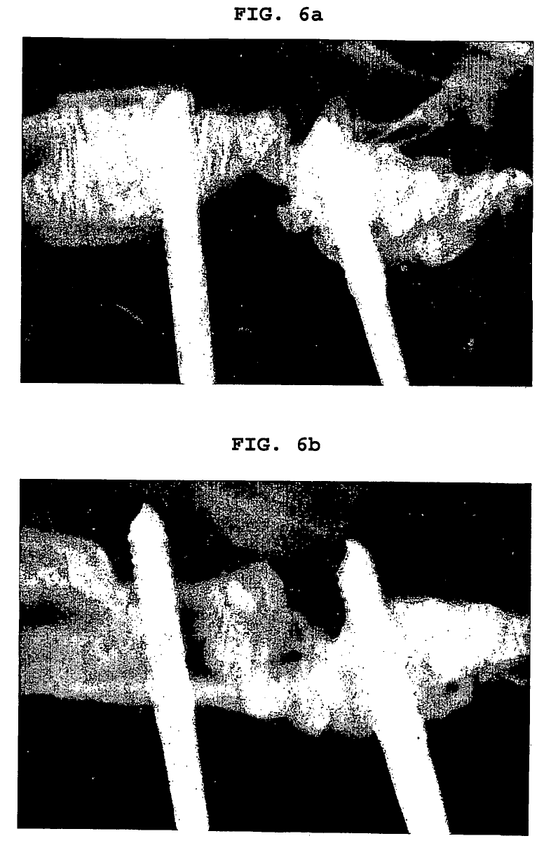

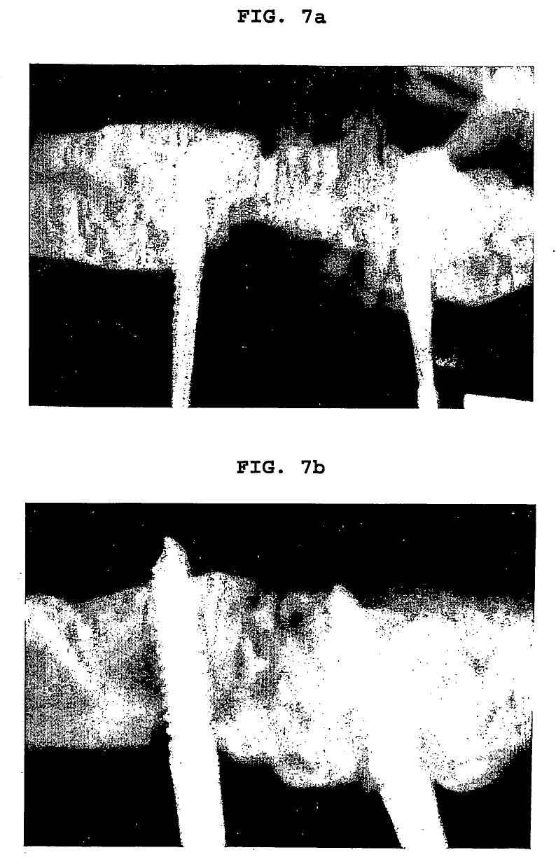

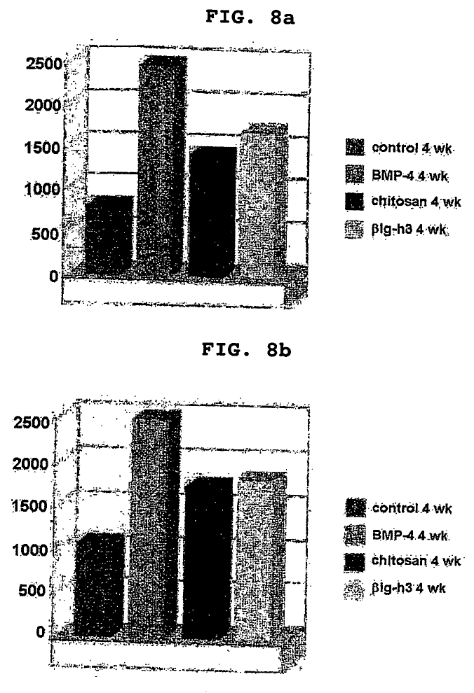

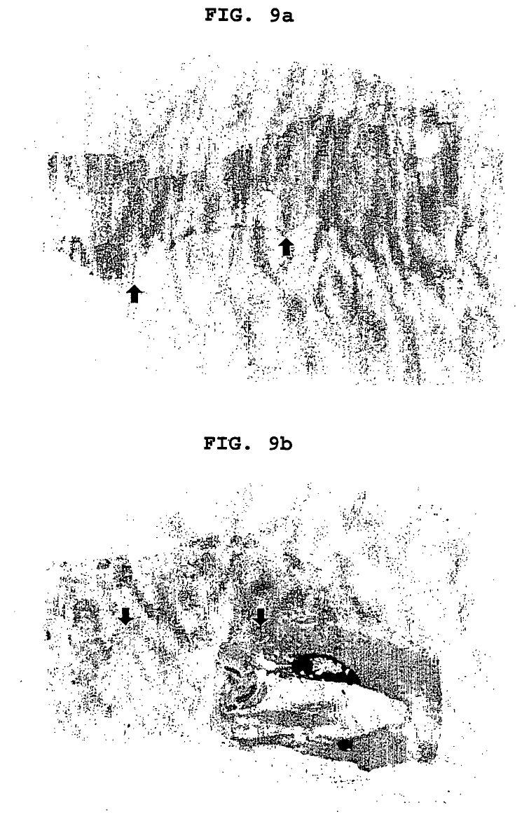

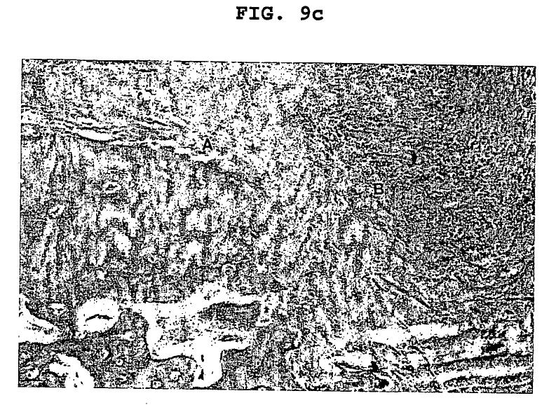

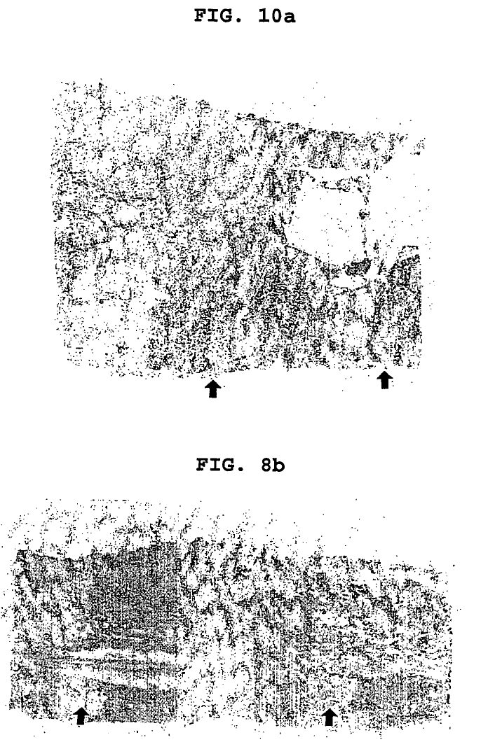

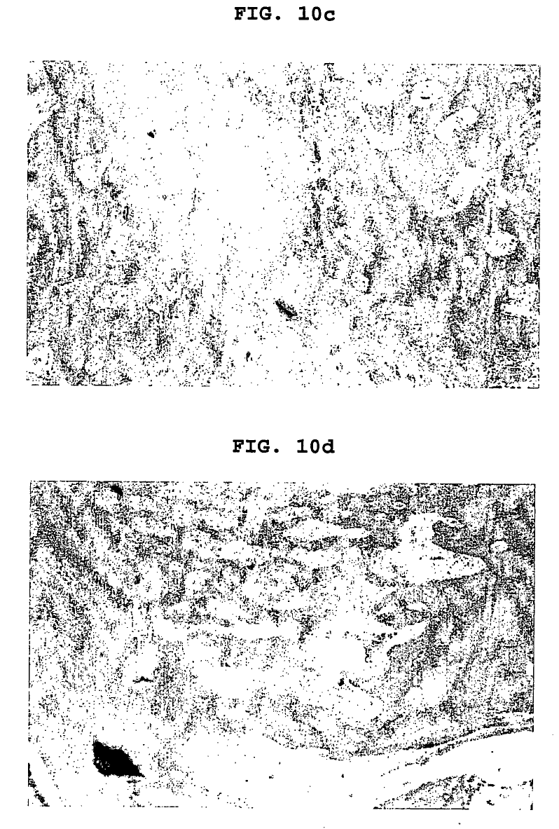

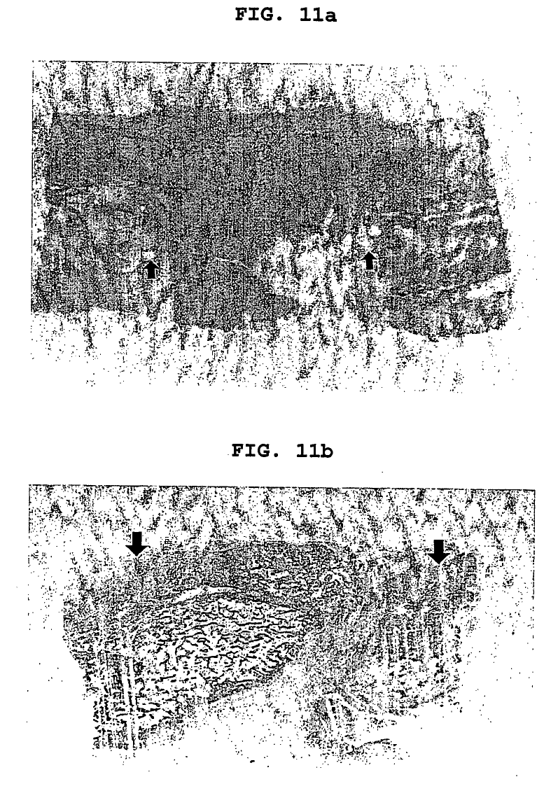

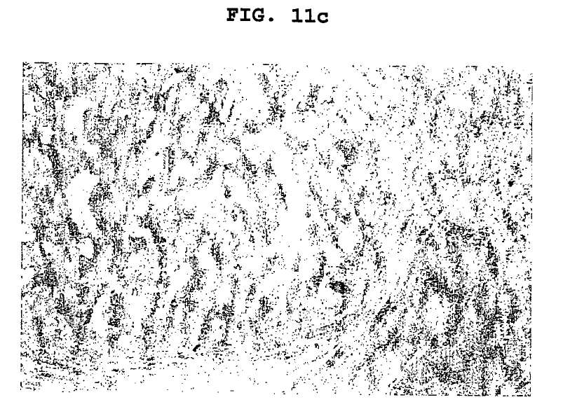

Radio-assay was performed for control group 4 weeks and 7 weeks after bone extension. As a result, wide radiolucent zone was found between extended jaw-spicules and radiodense zone abutting on jaw-spicules was hardly generated. The result obtained 4 weeks after bone extension was not very different from that obtained 7 weeks later in a control group. In the meantime, calcification was clearly observed at the extended area of jaw-bone wherein bone growth materials were introduced in BMP-4 group, β ig-h3 group and chitosan group. 4 weeks after introducing bone growth materials, it was confirmed by radio-assay that radiolucent zone between extende jaw-spicules was almost connected with radiodense zone growing from both sides of spicules. The thickness from top to bottom of radiodense zone became more than two fold 7 weeks later, comparing to that after 4 weeks. Especially, darker radiodense shadow and thicker radiodense zone were observed in the BMP-4 group, comparing to other groups (see FIG. 4-FIG. 7).

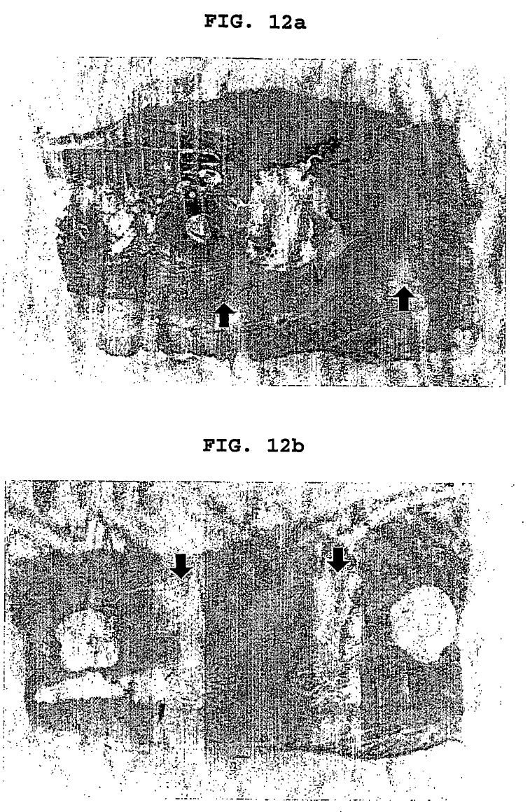

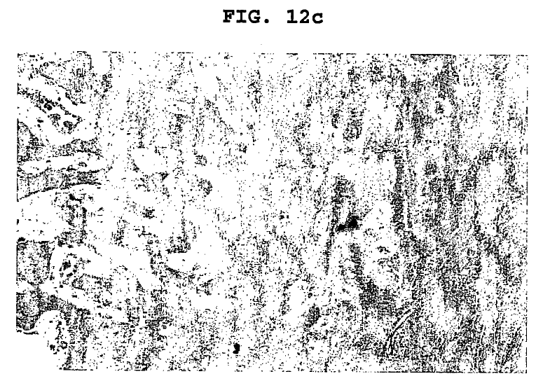

Besides, every group had higher bone-mineral density than control group. Especially, BMP-4 group showed the highest bone-mineral density and β ig-h3 group, chitosan group and control group followed in order. Bone-mineral density reflects radiodense level on extended area between jaw-spicules, meaning that the higher the density is the greater new bone-formation becomes (see FIG. 8).

Histological test was also performed. As a result, the whole bone-extended area was filled with fibrous tissue 4 weeks later in control group. Although new bone-formation close to jawbone section was begun by periosteum reaction, general bone-formation was not found (see FIG. 9A). 7 weeks later, however, new bone and cartilage-formation were detected near the edge of extended area and blood vessels and nervous tissues were also found in many places (see FIGS. 9B and 9C).

As for BMP-4 group, the proliferation of osteoblasts forming osteoid was partly observed in center and near the edge of extended spicules, but most parts of the extended area were filled with fibrous tissues 4 weeks later (see FIGS. 10A and 10C). 7 weeks later, irregular woven bone trabeculae was partially calcified at extended area, newly formed bone area wherein blood vessels in various sizes were spread and narrow fibrous interzone lying in the middle of the newly formed bone area were observed. The new bone formed throughout the whole extended area was similar to the normal cortical bone (see FIGS. 10B and 10D).

As for β ig-h3 group, osteoblasts forming osteoid were partly observed in the center of the extended area, which was confirmed by histological test 4 weeks after bone-extension (see FIG. 11A). 7 weeks later, lots of osteoblasts were partly forming new bone from the edge to the center of extended area. The amount of newly formed bone was smaller than that of BMP-4 group but fibrous interzone locating in the center of extended area was wider than that of BMP-4 group (see FIG. 11B and 11C).

As for chitosan group, histological test 4 weeks later confirmed that most extended area were filled with fibrous tissues (see FIG. 12A). 7 weeks later, lots of osteoblasts along with new bone were observed over the edge of extended area and new bones were confirmed to be formed partly form the edge to the center of extended area (see FIGS. 12B and 12C). The amount of newly formed bone in extended area was smaller than that of β ig-h3 group or BMP-4 group, but fibrous interzone was wider than that of β ig-h3 group.

Resultingly, the composition of the present invention which was prepared by adding water soluble chitosan, water soluble chitosan containing β ig-h3, and water soluble chitosan containing BMP-4 to tripolyphosphate can be effectively used for stimulating bone-formation and bone-consolidation by stimulating bone-formation and bone-consolidation in the early phase and by shortening bone-consolidation period.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation of Composition

The present inventors have prepared a composition for stimulating bone-formation and bone-consolidation by adding a material to stimulate bone-formation and bone-consolidation to tripolyphosphate. Particularly, the present inventors prepared a composition by adding 0.5 ml of 5% chitosan to 0.5 ml of 5% tripolyphosphate and named it “chitosan group”.

### Example 2

### Preparation of Composition

The present inventors prepared a composition for stimulating bone-formation and bone-consolidation by adding 0.5 Ml of water soluble chitosan containing β ig-h3 at the concentration of 450 μg/ml to 0.5 ml of 5% tripolyphosphate with the same method as the above Example 1 and named it “β ig-h3 group”.

### Example 3

### Preparation of Composition

The present inventors prepared a composition for stimulating bone-formation and bone-consolidation by adding 0.5 ml of water soluble chitosan containing BMP-4 at the concentration of 200 ng/ml to 0.5 ml of 5% tripolyphosphate with the same method as the above Example 1 and named it “BMP-4 group”.

### Experimental Example 1

### Investigating the Effect of the Compositions of the Present Invention on Bone-Formation and Bone-Consolidation

In order to confirm whether the compositions for stimulating bone-formation and bone-consolidation prepared through Example 1-Example 3 of the present invention can stimulate new bone-formation and bone-consolidation in the early phase, the present inventors first performed bone extension operation at jawbones of dogs, followed by injecting the compositions for stimulating bone-formation and bone-consolidation thereto, and then observed the changes occurring.

Particularly, used 16 5-8 month old dogs for experiments and grouped them by 4 for control group, chitosan group, β ig-h3 group and BMP-4 group.

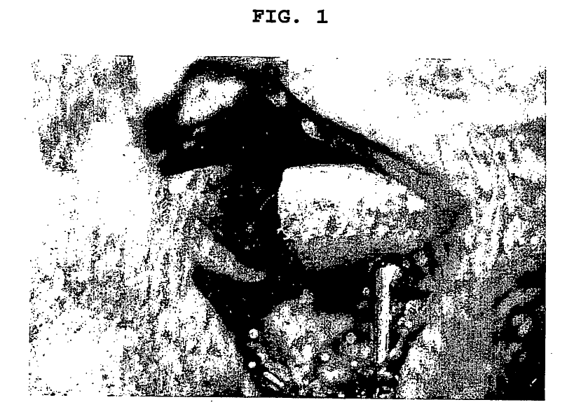

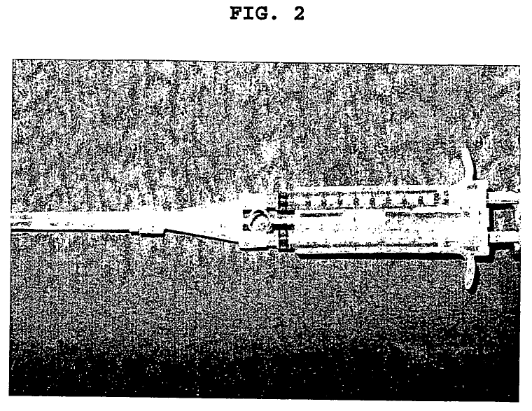

Kept the breath of dogs through tubes inserted in organs after general anesthesia and shaved the operating part, followed by sterilization and application. Incised skin 3-4 cm along the lower end of jawbone, lifted masseter muscle and exposed the side part of jawbone. Then, performed vertical osteotomy at the trunk of jawbone using an electric saw and completely cut jawbone. Fixed each fixing pin of external fixator on spicule 1 cm away from the cutting area right and left. While fixing the pins on the jawbone spicule with drill, kept washing with saline solution not to burn the fixing sites. Inserted the pins just as deep as it barely passed through jawbone and then fixed them tightly. After fixing two pins all, set them on bone extending apparatus (Molina Distractors, Wells Johnson Company) (FIG. 1).

Sutured the incised area with 5-0 vicryl and 5-0 nylon stitching fiber layer upon layer and recovered the dogs from anesthesia. Administered penicillin (100,000μ/kg) by intramuscular injection every 12 hours for 7 days after operation and oral-administered anodyne every 4-6 hours to relieve pain. Fed the dogs with soft diet until the second day after operation and provided regular diet from the third day. From the fifth day after operation, started bone-extension 2 mm per day for 5 days (up to 10 mm total).

On the day when bone extension was finished, injected 5% water soluble chitosan, 5% water soluble chitosan containing BMP-4(200 ng/ml) (R&D System Inc.) and 5% water soluble chitosan containing β ig-h3 (450 μg/ml) with the same amount as 0.5 ml of 5% tripolyphosphate(TPP) simultaneously to the same extended spot using a dual syringe (FIG. 2) for each group to induce immediate hardening by mixing those materials, resulting in the prevention of transferring those injected materials to other areas for strong new bone formation. On the contrary, injected only 1 ml of 5% tripolyphosphate to a control group.

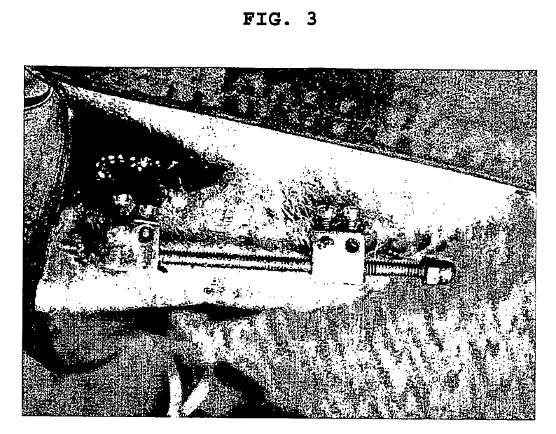

Upon completing the injection of compositions for stimulating bone-formation and bone-consolidation of the present invention to the dogs, kept bone-extending apparatus on them for 7 weeks for bone-consolidation and bone-regeneration. Sacrificed every 2 dogs from each group by injecting overdose of pentobarbital (40-50 mg/kg) 4 weeks after bone extension, so did for the rest 8 dogs 7 weeks after bone extension (FIG. 3).

<1-1> Observation with the Naked Eye

The changes after the above experiments were observed with the naked eye. Every dogs were well recovered from anesthesia and operation, bone-extending apparatus was kept stable and no signs of infection around the apparatus were shown. Bone samples taken 4 weeks and 7 weeks after bone extension confirmed that the extended bone area was hardened but a little flexible when being bended in a control group. Meanwhile, 4 week old extended bone areas of BMP-4 group, β ig-h3 group and chitosan group were harder than 7 week old extended bone area of a control group and all the groups except the control group showed very strong and solid new bone formation 7 weeks later.

<1-2> Radio-Assay

Examined each animal group went through bone extension with radio-assay every week and observed bone-formation and bone-consolidation through the radiographs taken after 4 weeks and 7 weeks respectively.

As a result, wide radiolucent zone between extended jawbone spicules was seen in a control group both 4 weeks and 7 weeks after bone extension was completed while radiodense zone close to both sides of jawbone spicules was hardly seen 4 weeks later having no difference in result obtained 7 weeks after bone extension. In the meantime, calcification was undergoing with the lapse of time in extended area of jawbone in every BMP-4 group, β ig-h3 group and chitosan group whereto appropriate material for bone growth was injected. As for those groups, radiolucent zone in between extended spicules was almost connected with radiodense zone growing from each side of spicules, which was confirmed by radiographs taken on the 4^{th} week. On the 7^{th} week, the thickness of radiodense zone became two-fold comparing with that of the 4^{th} week. Especially, the density of radiodense shadow in BMP-4 group was thicker and darker comparing to other groups.

<1-3> Measurement of Bone Mineral Density

Based on radiographs (FIG. 4B, 5B, 6B and 7B) taken on the 7^{th} week in the above Example <1-2>, the present inventors measured bone mineral density with computer program by taking advantage of the fact that radiodense zone becomes brighter on radiograph as bone-formation progresses.

As a result, bone mineral density in those groups was higher than that in control group on the 4^{th} week. Especially, BMP-4 group showed the highest density and β ig-h3 group, chitosan group and control group followed in order (FIG. 8A). BMP-4 group still showed remarkably high density comparing to other groups on the 7^{th} week and β ig-h3 group, chitosan group and control group also followed in order (FIG. 8B). Bone mineral density reflects the brightness on a radiograph, in other words, the degree of radiodense in extended area between jawbone spicules. Thus, the higher the density, the greater the amount of bone-formation is. Resultingly, it was confirmed that the groups whereto compositions for stimulating bone-formation and bone-consolidation were added showed faster and greater bone-formation than a control group.

<1-4> Histological Test

Bone samples were taken from extended jawbone including normal osseous tissues therearound with an electric saw. The obtained spicules were fixed in 10% neutral formalin for 1 week, and then decalcified in 10% nitric acid and 10% sodium citrate for 2 days. 4-6 μm of specimens were prepared by dehydration and paraffin-fixation following general techniques. The specimens were stained with hematoxylin-eosin to observe histological changes with an optical microscope.

As a result, it was observed in the control group that the whole extended bone area was filled with fibrous tissues and general new bone-formation was not detected even though new bone-formation by periosteum reaction had just begun along the section of jawbone on the 4^{th} week after bone extension (FIG. 9A). New bone and cartilage were formed over the edge of extended area and blood vessels and nervous tissues were also observed in many places (FIGS. 9B and 9C).

As for BMP-4 group, although the proliferation of osteoblasts forming osteoid was partly observed in the center and through the edge of extended spicules, most parts of the extended area were filled with fibrous tissues on the 4^{th} week (FIGS. 10A and 10C). On the 7^{th} week, widely formed new bone area having irregular woven bone trabeculae resulted from partial calcification, various sized blood vessels and narrow fibrous interzone lying up and down in the center of new bone area were observed. The new bone formed throughout the whole extended area was almost similar to the normal cortical bone (FIG. 10B and 10D).

As for β ig-h3 group, it was observed on the 4^{th} week that osteoblasts forming osteoid were partly proliferated in the center of the extended area (FIG. 11A). On the 7^{th} week, many active osteoblasts were partly forming new bone from the edge through the center of the extended area, but the volume of new bone was smaller than that of BMP-4 group. The fibrous interzone lying up and down in the center of the extended area was, though, wider than that of BMP-4 group (FIG. 11B and 11C).

As for chitosan group, most parts of the extended area were filled with fibrous tissues on the 4^{th} week (FIG. 12A). On the 7^{th} week, lots of osteoblasts along with new bone were observed over the edge of the extended area and also new bone was partly formed from the edge through the center of the extended area. The volume of newly formed bone was smaller than those of β ig-h3 group and BMP-4 group but the fibrous interzone was wider than that of β ig-h3 group (FIGS. 12B and 12C)

Resultingly, as for BMP-4 group, β ig-h3 group and chitosan group treated with the composition for stimulating bone-formation and bone-consolidation of the present invention, new bone was partly formed in extended area on the 4^{th} week after bone extension and the formation went further on the 7^{th} week. BMP-4 group had the biggest volume of newly formed bone and β ig-h3 group, chitosan group followed in order. Fibrous interzone was found in the center of the extended area in every group on the 7^{th} week. The fibrous interzone of BMP-4 group was the narrowest, β ig-h3 group showed the second narrowest fibrous interzone and chitosan group was the last. In the meantime, the fibrous interzone took most parts of the extended area in the control group. Observing fibrous interzone still on the 7^{th} week suggests that new-bone formation is still undergoing.

### INDUSTRIAL APPLICABILITY

As described hereinbefore, the composition of the present invention prepared by adding a material for stimulating bone-formation and bone-consolidation to the mixture of tripolyphosphate and water-soluble chitosan can be effectively used for stimulating bone-formation and bone-consolidation. Precisely, the composition induces new bone-formation, provides a normal bone-structure, prevents the growth of unnecessary connecting tissues and is suitable enough for human to supplement bone-loss during the recovery process as well as induces the growth of blood vessels and bony osteogenesis cells in the early stage.

What is claimed is:

1. A composition containing tripolyphophate and water-soluble chitosan for stimulating bone-formation and bone-consolidation.

2. The composition for stimulating bone-formation and bone-consolidation as set forth in claim 1, wherein the composition could additively contain a material for stimulating bone-formation and bone-consolidation.

3. The composition for stimulating bone-formation and bone-consolidation as set forth in claim 2, wherein the material for stimulating bone-formation and bone-consolidation is selected from a group consisting of β ig-h3, bone morphogenic protein, TGF-β, FGF, IGF-1 and PDGF.

4. The composition for stimulating bone-formation and bone-consolidation as set forth in claim 1, wherein the ratio of tripolyphosphate to water-soluble chitosan is 20:80˜80:20 weight %.

5. The composition for stimulating bone-formation and bone-consolidation as set forth in claim 4, wherein the ratio of tripolyphosphate to water-soluble chitosan is 50:50 weight %.

6. The composition for stimulating bone-formation and bone-consolidation as set forth in claim 3, wherein the β ig-h3 is added at the concentration of 100 μg/ml˜1 μg/ml.

7. The composition for stimulating bone-formation and bone-consolidation as set forth in claim 3, wherein the bone morphogenic protein is added at the concentration of 50 ng/ml˜500 ng/ml.

8. The composition for stimulating bone-formation and bone-consolidation as set forth in claim 6, wherein the β ig-h3 is added at the concentration of 300 μg/ml˜600 μg/ml.

9. The composition for stimulating bone-formation and bone-consolidation as set forth in claim 7, wherein the bone morphogenic protein is added at the concentration of 100 ng/ml˜300 ng/ml.

10. The composition for stimulating bone-formation and bone-consolidation as set forth in claim 4, wherein the bone morphogenic protein is BMP-4.
